(19) United States
(12) Patent Application Publication
(10) Pub. No.: US 2022/0011311 A1
(43) Pub. Date: Jan. 13, 2022

(54) DETECTION OF BLADDER CANCER

(71) Applicant: Randox Laboratories Ltd., Northern Ireland (GB)

(72) Inventors: Mark Ruddock, Northern Ireland (GB); John Lamont, Northern Ireland (GB); Stephen Fitzgerald, Northern Ireland (GB); Kathleen Williamson, Belfast, Northern Ireland (GB)

(21) Appl. No.: 17/294,358

(22) PCT Filed: Nov. 15, 2019
(86) PCT No.: PCT/GB2019/053253

§ 371 Date: May 14, 2021

(30) Foreign Application Priority Data
Nov. 16, 2018 (GB) 1818744.3

(51) Int. Cl. G01N 33/574

(52) U.S. Cl. CPC G01N 33/57407

(57) ABSTRACT

The present invention provides a method for detecting the presence or risk of bladder cancer in a female patient comprising the steps of detecting the presence of a panel of biomarkers in a sample isolated from a female patient, said panel of biomarkers comprising IL-13 and IL-12p70 and one or more biomarkers selected from BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF and Triglycerides and/or the concentration of albumin/microalbumin/protein and creatinine expressed as an albumin:creatinine ratio in a sample isolated from a female patient; and assessing the results and comparing them to a normal control wherein an elevated presence of the biomarker compared to a normal control indicates the presence or risk of cancer in the patient from whom the sample is isolated.

### FIELD OF INVENTION

The invention relates to a method of detecting the presence of, or the risk of, bladder cancer in a female patient.

### BACKGROUND OF THE INVENTION

Bladder cancer is a leading cause of death worldwide. Bladder cancer is more than three times more common in men than women though the mortality rate in the latter is twice as great. Female bladder cancer patients in England and Wales have almost 20% lower survival rates at 1 and 5 years, and almost 30% at 10 years, suggesting that female patients are presenting with a more advanced disease. A multivariate analyses controlling for sex and race found that 39% of men with haematuria were referred to a urologist by their GP, compared with 17% of women with haematuria. Furthermore, men were more likely than women to have a complete evaluation (22% vs. 12%) and less likely to have an incomplete evaluation (55% vs. 69%) for their haematuria.

The usefulness of a diagnostic test is measured by its sensitivity and specificity. The sensitivity of a test is the number of true positives (the number of individuals with a particular disease who test positive for the disease) and the specificity is the number of true negatives (the number of individuals without a disease who test negative for the disease). The most common sign of bladder cancer is gross or microscopic haematuria, often detected by the family physician, and is observed in 85% of all bladder cancer patients. A simple urine dip test can be used to detect the presence of blood. Although cancer without blood is rare, leading to high sensitivity of a simply blood dip test, the specificity of the test is poor with fewer than 5% of patients presenting with haematuria actually having bladder cancer. However, the 5% of patients who do present are normally diagnosed with superficial tumours, which can easily be resected.

Cystoscopy and cytology are the preferred methods used to diagnose bladder cancer. A cytological examination involves the examination of urothelial cells in voided urine. This method has high specificity and it is convenient to obtain a sample. However, it has poor sensitivity and is subjective at low cellular yield. A cytological assessment is usually combined with flexibly cystoscopy. White light cystoscopy (WLC) allows direct observation of the bladder and biopsy of suspicious regions. However, recent publications have shown that blue light cystoscopy (BLC) picked up 34% more tumours (e.g. carcinoma in situ (CIS)) that WLC. Furthermore, 20/53 patients (37.7%) with CIS lesions had negative cytology (Fradet et al., 2007; Witjes et al., 2010). Unfortunately, BLC has a higher false positive rate than WLC (39% vs. 31%, respectively) (Fradet et al., 2007). Cystoscopy has a sensitivity and specificity of 71% and 72%, respectively (National Collaborating Centre for Cancer, Bladder Cancer: diagnosis and management; NICE Guideline 2, February 2015, Page 78).

There are some disadvantages associated with cystoscopy, namely that it is expensive, causes patient discomfort, risk of infection and does not allow for upper tract visualization or for the detection of small areas of CIS e.g. increased number of bladder cancer recurrences detected on cystoscopy when information on a positive urine test (cytology) is communicated to the urologist; but not when the result is blinded (van der Aa et al., 2010).

Attempts have been made in the art to identify one or more biochemical bladder cancer biomarkers that could identify patients who present with bladder cancer before committing them to cystoscopy. At the present time approximately 20% patients present with advanced disease and their prognosis is poorer as a result. Attempts have therefore been made in the art to identify a proven biomarker or panel of biomarkers, which could be used as a screening tool for bladder cancer, particularly for low-risk asymptomatic patients.

No single biomarker or panel of biomarkers has yet achieved the levels of sensitivity and specificity required to reduce the frequency of cystoscopy needed for an accurate diagnosis. Over the last 10 years a large number of bladder cancer markers including Bladder Tumour Antigen (BTA), Nuclear Matrix Protein 22 (NMP22), telomerase and fibrinogen-degradation product(s)(FDP), have been evaluated against the gold standard urine cytology with quite consistent results of low specificity. These markers are present in urine in a large proportion of patients with urological pathologies other than bladder cancer and in patients with urinary infections (UTIs). NMP22 and BTA have FDA approval as point of care assays. However, NMP22 requires immediate stabilization in urine, which is not always possible, and BTA can be confounded by blood present in the urine. New putative markers, such as survivin, hyaluronic acid, cytokeratin 8 and 18 and EGF, which have been shown to induce expression of the matrix metalloproteinase (MMP9) in some bladder cancer cells, have been proposed as bladder cancer markers. However, none of the putative biomarkers have been bench-marked against the high specificity of urine cytology and the high sensitivity of the telomerase assay.

Thus, in the field of bladder cancer diagnosis and treatment, the biomarkers identified in the prior art are unsatisfactory since they lack the required sensitivity and specificity required to make an accurate diagnosis of bladder cancer or assessment of a patient's risk in developing the disease. As a result, the clinician is not able to accurately assess whether a patient should be put forward for further cytoscopic and cytological tests which results in high costs associated with diagnosing and managing the disease.

A lot of money and resources are used on giving low-risk patients cystoscopies who could actually be managed in primary care rather than increasing the wait for high-risk patients to get a cystoscopy. There is therefore a need for a test that provides an accurate assessment which can allow a GP to rule out bladder cancer from the diagnosis without sending the patient for a cystoscopy.

### SUMMARY OF THE INVENTION

The present invention is based on the realization that there are significant differences between the biomarkers required in the diagnosis of bladder cancer in males and females. The present invention therefore provides specific panels of biomarkers useful in the diagnosis of bladder cancer in female subjects.

In a first aspect of the invention there is a method for the detection of or the risk of bladder cancer in a female patient comprising the steps of

(i) detecting the presence of a panel of biomarkers in a sample isolated from a female patient, said panel of biomarkers comprising IL-13 and IL-12p70 and one or more biomarkers selected from BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF and Triglycerides and/or the concentration of albumin/microalbumin/protein and creatinine expressed as an albumin:creatinine ratio;

(ii) assessing the presence or risk of bladder cancer in the female patient wherein detection of an elevated presence of the biomarkers compared to a normal control indicates the presence or the risk of cancer in the female patient from whom the sample is isolated.

In a second aspect of the invention there is a solid support material comprising binding molecules attached thereto, said binding molecules having affinity specific for IL-13 and, separately, IL-12p70, with the binding molecules for each being in discrete locations on the support material.

In a third aspect of the invention there is a method for the detection of or the risk of bladder cancer in a female patient comprising the steps of

(i) determining that a female patient does not have an infection;

(ii) detecting the presence of one or more biomarkers in a sample isolated from the female patient, wherein said one or more biomarkers are selected from IL-13, IL12p70, BTA and Midkine;

(iii) assessing the presence or risk of bladder cancer in the female patient wherein detection of an elevated presence of the biomarkers compared to a normal control indicates the presence or the risk of cancer in the female patient from whom the sample is isolated.

### BRIEF DESCRIPTION OF THE FIGURES

The present invention is described with reference to the accompanying drawings, wherein:

FIG. 1: Shows the ROC curve outputted from the SPSS Analysis (HaBio) for Females (4 Biomarkers);

FIG. 2: Shows the ROC curve outputted from the SPSS Analysis (HaBio) for Females (4 Biomarkers+infection); and

FIG. 3: Shows the population pyramid count for all cancers by infection.

### DESCRIPTION OF THE INVENTION

The present invention is based on the finding that certain biomarkers present in a female patient suffering from bladder cancer, enable a more accurate diagnosis to be made compared to the prior art methods of diagnosis on the basis of biomarkers that are used for the diagnosis of men and women. Identification of particular biomarkers in a sample isolated from a female patient is indicative of the susceptibility to or the presence of cancer in the female patient, and it has been surprisingly found that these biomarkers differ significantly in men and women.

As used herein, the term ‘biomarker’ refers to a molecule present in a biological sample obtained from a patient, the concentration of which in said sample may be indicative of a pathological state. Various biomarkers that have been found to be useful in diagnosing bladder cancer, either alone or in combination with other diagnostic methods, or as complementary biomarkers in combination with other biomarkers, are described herein.

Diagnosis may be made on the basis of the level of expression or the concentration of the biomarker in a female patient isolated from the patient. The biomarkers of the present invention are typically identified in a serum or urine sample from the patient. Preferably, the sample is a urine sample.

The panel of biomarkers with which the present invention is concerned comprises IL-13 and IL-12p70 and one or more biomarkers selected from BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF, Triglycerides, preferably BTA, Midkine, PAI-1/tPA, Clusterin, IL-8, Microalbumin, MMP9NGAL, NSE, Cystatin C, d-Dimer, IL-7, and/or the concentration of albumin/microalbumin/protein and creatinine expressed as an albumin:creatinine ratio (ACR).

The panel of biomarkers may be any of the combinations listed in Table 2 or Table 3.

Preferably the panel of biomarkers is (i) BTA, IL-13 and IL12p70, (ii) Midkine, IL-13 and IL12p70, (iii) BTA, IL-13, IL12p70 and Midkine; or (iv) BTA, IL-13, IL12p70, Midkine and PAI-1/tPA.

In some embodiments the sample has a concentration of albumin and creatinine expressed as an albumin:creatinine ratio. This may be calculated by measuring the concentration separately of albumin and creatinine. The skilled person will appreciate conventional ways to measure albumin and creatinine concentrations, see examples for illustrative methods. When the kidneys are functioning properly there is virtually no albumin present in the urine.

In some embodiments the patient may be presenting with haematuria and/or with an infection. For the avoidance of doubt, the term ‘haematuria’ refers to the presence of red blood cells in the urine. Suitably the infection may be a bacterial or viral infection, preferably a bacterial infection. Suitably, the method may further comprise a step of characterizing the patient's infection status. Characterizing infection means diagnosing the patient as having infection or being infection free, and may include identifying the infecting species. Infection may be determined using clinical-based diagnoses based on clinical history, biomarkers, dipstick analysis or UTI multiplex array (e.g. Randox Urinary Track Multiplex Assay). By incorporating an initial infection test the AUCs can be increased and also reduce the number of biomarkers used to diagnose the bladder cancer. In the context of the present invention, the term ‘bladder cancer’ is understood to include urothelial carcinoma (UC), transitional cell carcinoma, bladder squamous cell carcinoma and/or bladder adenocarcinoma. In some embodiments the presence of haematuria and/or an infection may further increase the elevated levels of the biomarkers within the panel of biomarkers compared to if haematuria and/or the infection were not present in female bladder cancer patients.

Preferably the biomarkers are in the urinary form i.e. are identified in a urine sample.

In a preferred embodiment, the biomarkers within the panel may be identified and their concentrations within the sample determined either sequentially or simultaneously in the sample isolated from the patient. The biomarkers may be identified and their concentrations within the isolated sample may be determined by routine methods, which are known in the art, such as by contacting the sample with a substrate having binding molecules specific for each of the biomarkers included in the panel of biomarkers. Preferably the substrate has at least two binding molecules immobilized thereon, more preferably three, four or more binding molecules, wherein each binding molecule is specific to an individual biomarker and the first probe is specific for IL-13 and the second probe is specific to IL-12p70. As used herein, the term ‘specific’ means that the binding molecule binds only to one of the biomarkers of the invention, with negligible binding to other biomarkers of the invention or to other analytes in the biological sample being analyzed. This ensures that the integrity of the diagnostic assay and its result using the biomarkers of the invention is not compromised by additional binding events.

The biomarker concentrations may be measured by using methodology based on immuno-detection. As such, the binding molecule is preferably an antibody, such as a polyclonal antibody or a monoclonal antibody. As used herein, the term ‘antibody’ includes any immunoglobulin or immunoglobulin-like molecule or fragment thereof, Fab fragments, ScFv fragments and other antigen binding fragments. The term ‘polyclonal antibodies’ refers to a heterogeneous population of antibodies which recognize multiple epitopes on a target/antigen. The term ‘monoclonal antibodies’ refers to a homogenous population of antibodies (including antibody fragments), which recognize a single epitope on a target/antigen. Immuno-detection technology is also readily incorporated into transportable or hand-held devices for use outside of the clinical environment. A quantitative immunoassay such as a Western blot or ELISA can be used to detect the amount of protein biomarkers. A preferred method of analysis comprises using a multi-analyte biochip which enables several proteins to be detected and quantified simultaneously. 2D Gel Electrophoresis is also a technique that can be used for multi-analyte analysis.

In a preferred embodiment, the binding molecules are immobilized on a solid support, ready to be contacted with the patient sample. A preferred solid support material is in the form of a biochip. A biochip is typically a planar substrate that may be, for example, mineral or polymer based, but is preferably ceramic. The solid support may be manufactured according to the method disclosed in, for example, GB-A-2324866 the contents of which is incorporated herein in its entirety. The solid supports may be screen printed in accordance with known methods disclosed in, for example, WO2017/085509. Preferably, the Biochip Array Technology system (BAT) (available from Randox Laboratories Limited) may be used to determine the levels of biomarkers in the sample. More preferably, the Evidence Evolution and Evidence Investigator apparatus (available from Randox Laboratories) may be used.

The solid support material comprises binding molecules attached thereto, said binding molecules having affinity specific for IL-13 and, separately, IL-12p70, with the binding molecules each being in discrete locations on the support material. The solid support material may further comprise, each in discrete locations, one or more binding molecules each having affinity specific for an additional biomarker selected from BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF and Triglycerides, preferably BTA, Midkine, PAI-1/tPA, Clusterin, IL-8, Microalbumin, MMP9NGAL, NSE, Cystatin C, d-Dimer and IL-7. For example, the binding molecules attached to the solid support material may have affinities to the combinations of biomarkers in Table 2 or Table 3, preferably (i) BTA, IL-13 and IL12p70, (ii) Midkine, IL-13 and IL12p70, (iii) BTA, IL-13, IL12p70 and Midkine; or (iv) BTA, IL-13, IL12p70, Midkine and PAI-1/tPA.

The present invention also provides the use of the substrate described in a method for the detection of or the risk of bladder cancer in a female patient.

The present invention also provides kits comprising probes for a panel of biomarkers comprising IL-13 and IL-12p70 and one or more biomarkers selected from BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF and Triglycerides preferably BTA, Midkine, PAI-1/tPA, Clusterin, IL-8, Microalbumin, MMP9NGAL, NSE, Cystatin C, d-Dimer and IL-7, and optionally reagents for the measurement of albumin and creatinine. For example, the panel of biomarkers may be the combinations in Table 2 or Table 3, preferably (i) BTA, IL-13 and IL12p70, (ii) Midkine, IL-13 and IL12p70, (iii) BTA, IL-13, IL12p70 and Midkine; or (iv) BTA, IL-13, IL12p70, Midkine and PAI-1/tPA. Such kits can be used to detect bladder cancer or the risk of bladder cancer in a female patient according to the first aspect of the invention.

The invention also provides a method for the detection of or the risk of bladder cancer in a female patient comprising the steps of

(i) determining that a female patient does not have an infection;

(ii) detecting the presence of one or more biomarkers in a sample isolated from the female patient, wherein said one or more biomarkers are selected from IL-13, IL12p70, BTA and Midkine;

(iii) assessing the presence or risk of bladder cancer in the female patient wherein detection of an elevated presence of the biomarkers compared to a normal control indicates the presence or the risk of cancer in the female patient from whom the sample is isolated.

Suitably, the one or more biomarkers are (i) IL-13+IL12p70, (ii) IL-13+BTA; (iii) IL-13+Midkine; (iv) IL12p70+BTA; (v) IL12p70+Midkine; or (vi) BTA+Midkine.

In the methods of the present invention, in order for bladder cancer or the risk of bladder cancer to be diagnosed, the biomarkers within the panel of biomarkers tested may be found at an elevated level compared to the corresponding biomarker in a normal control sample. In some embodiments, the concentrations of biomarkers are found at a significantly higher level than in a control sample. The determination of “higher concentration” is relative and determined with respect to a control subject known not to have bladder cancer.

Control values are derived from the concentration of corresponding biomarkers in a biological sample obtained from an individual or individuals who do not have bladder cancer. Such individual(s) may be, for example, healthy individuals or individuals suffering from diseases other than bladder cancer. Alternatively, the control values may correspond to the concentration of each of the biomarkers in a sample obtained from the patient prior to getting bladder cancer.

For the avoidance of doubt, the term ‘corresponding biomarkers’ means that concentrations of the same combination of biomarkers that are determined in respect of the patient's sample are also used to determine the control values. For example, if the concentration of IL-13 and IL-12p70 in the patient's sample is determined, then the concentration of IL-13 and IL-12p70 in the control is also known.

In a preferred embodiment, each of the female patient and control biomarker concentration values is inputted into one or more statistical algorithms to produce an output value that indicates whether bladder cancer is present in the patient. If the output value is less than the biomarker cut-off, the patient is negative by biochip for bladder cancer. If the output value is higher than the biomarker cut-off, the patient is positive by biochip for bladder cancer.

In a preferred embodiment, a Clinical Risk Score (CRS) is calculated for the female patient, which is a cumulative score using, but not restricted to, the following clinical and demographic measurements: age, haematuria (non-visible vs. macro haematuria), smoking (pack years), BMI, blood pressure (controlled, normotensive, hypertensive), occupational risk score (FINJEM), social class (ONS Codes), comorbidities e.g. diabetes, chronic kidney disease (CKD) etc., medications e.g. statins, anti-hypertensives etc, specific medications (found to increase risk of bladder cancer), pain relief, renal transplant, kidney cancer, other cancers, pelvic radiotherapy and UTIs (with/without microbiology).

Example scores used when calculating the CRS for a patient: age is greater than 65 equals a score of 1; age is less than 65 equals a score of 0; non-visible haematuria (NVH) equals a score of 1; macro haematuria equals a score of 2. Therefore, a patient who is older than 65 years with macro haematuria would have a cumulative score of 3, using age and haematuria as clinical risk scores.

In a preferred embodiment, the biochip bladder cancer test data and CRS is combined to determine if the patient was in one of the following categories: low risk, medium risk or high risk. This information would allow the GP to manage his/her patients in primary care and refer them to further tests if and when appropriate. For example, patients who present with haematuria and are negative by biochip and have a low CRS would be monitored in primary care by their GP, rather than being referred to have a cystoscopy. Patients who are negative by biochip and have a moderate CRS would be referred to urology for cystoscopy (non-urgent). Patients who are positive by biochip and have a low CRS would be referred to urology for cystoscopy (non-urgent). Patients who are positive by biochip and have moderate CRS would be ‘red flagged’ for an urgent cystoscopy.

| | Bladder Cancer Test | | Clinical Risk | Score (CRS) |
|---|---|---|---|---|
| | Negative | Positive | Low | Moderate |
| Monitor at 6 & 12 | Y | N | Y | N |
| months using the | | | | |
| Test and CRS | | | | |
| Referral for | Y | N | N | Y |
| Cystoscopy | | | | |
| (non-urgent) | | | | |
| Referral for | N | Y | Y | N |
| Cystoscopy | | | | |
| (non-urgent) | | | | |
| Urgent referral for | N | Y | N | Y |
| Cystoscopy | | | | |
| (urgent) | | | | |

Key: Y = Yes; N = No

The accuracy of statistical methods used in accordance with the present invention can be best described by their receiver operating characteristics (ROC). The ROC curve addresses both the sensitivity, the number of true positives, and the specificity, the number of true negatives, of the test. Therefore, sensitivity and specificity values for a given combination of biomarkers are an indication of the accuracy of the assay. For example, if a biomarker combination has sensitivity and specificity values of 80%, out of 100 patients which have bladder cancer, 80 will be correctly identified from the determination of the presence of the particular combination of biomarkers as positive for bladder cancer, while out of 100 patients who have not got bladder cancer 80 will accurately test negative for the disease.

The ROC also provides a measure of the predictive power of the test in the form of the area under the curve (AUC). AUC is a measure of the probability that the perceived measurement will allow correct identification of a condition. By convention, this area is always 0.5. Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). As a general rule, a test with a sensitivity of about 80% or more and a specificity of about 80% or more is regarded in the art as a test of potential use, although these values vary according to the clinical application. In a preferred embodiment, the panel of biomarkers has an AUC value of at least 0.7, suitably at least 0.75, preferably at least 0.8, more preferably at least 0.85.

It is well understood in the art that biomarker normal or ‘background’ concentrations may exhibit slight variation due to, for example, age, gender or ethnic/geographical genotypes. As a result, the cut-off value used in the methods of the invention may also slightly vary due to optimization depending upon the target patient or population. Adjusting the cut-off will also allow the operator to increase the sensitivity at the expense of specificity and vice versa.

In one embodiment, the algorithm has a sensitivity and/or specificity of at least 0.7 respectively. Preferably, the algorithm has a sensitivity of at least 0.75, more preferably of at least 0.8, and/or a specificity of at least 0.75, more preferably of at least 0.8.

Where two or more biomarkers are used in the invention, a suitable mathematical or machine learning classification model, such as logistic regression equation, can be derived. The skilled statistician will understand how such a suitable model is derived, which can include other variables such as age and gender of the patient. The ROC curve can be used to assess the accuracy of the model, and the model can be used independently or in an algorithm to aid clinical decision making. Although a logistic regression equation is a common mathematical/statistical procedure used in such cases and an option in the context of the present invention, other mathematical/statistical, decision trees or machine learning procedures can also be used. The skilled person will appreciate that the model generated for a given population may need to be adjusted for application to datasets obtained from different populations or patient cohorts.

The following examples illustrate the invention with reference to the figures.

### EXAMPLES

### Patients

One hundred and fifty-seven patients presenting with haematuria were recruited for a bladder cancer trial. Having established the feasibility of diagnostic algorithms for bladder cancer in haematuria patients, a large Haematuria Biomarker Study (HaBio) was designed which recruited six hundred and seventy-five patients.

### Urine & Serum Collection

Urine samples (˜50 ml) and serum samples (˜10 ml) were collected from all patients in sterile containers. Unfiltered and uncentrifuged urine samples were immediately aliquoted and frozen at −80° C. until analyses. Urine samples were thawed on ice and then centrifuged (1200×g, 10 minutes, 4° C.) to remove any particulate matter prior to analysis.

### Biomarker Measurement

All samples were run in triplicate and the results are expressed as mean±SD (n=3).

Biochip Array Technology (Randox Laboratories Ltd., Crumlin, Northern Ireland, UK) was used for the simultaneous detection of multiple analytes from a single patient samples (urine). The technology is based on the Randox Biochip, a 9 mm² solid substrate supporting an array of discrete test regions with immobilized, antigen-specific antibodies. Following antibody activation with assay buffer, standards and samples were added and incubated at 37° C. for 60 minutes, then placed in a thermo-shaker at 370 rpm for 60 minutes. Antibody conjugates (HRP) were added and incubated in the thermo-shaker at 370 rpm for 60 minutes. The chemiluminescent signals formed after the addition of luminol (1:1 ratio with conjugate) were detected and measured using digital imaging technology and compared with that from a calibration curve to calculate concentration of the analytes in the samples. The analytical sensitivity of the biochip was as follows: IL-2 4.8 pg/ml, IL-4 6.6 pg/ml, IL-6 1.2 pg/ml, IL7 1.11 pg/ml, IL-8 7.9 pg/ml, IL12p70 2.61 pg/ml, IL-13 5.23 pg/ml, VEGF 14.6 pg/ml, TNFα 4.4 pg/ml, IL-1α 0.8 pg/ml, IL-1β 1.6 pg/ml, MCP-1 13.2 pg/ml, NSE 0.26 ng/ml, NGAL 17.8 ng/ml, sTNFRI 0.24 ng/ml, d-Dimer 2.1 ng/ml, sTNFRII 0.2 ng/ml. Functional sensitivity for CEA and PSA (free and total) were 0.2, 0.02 and 0.045 ng/ml, respectively. Data below the Limit of Detection (LOD)/Mean Detectable Dose (MDD)—when data was below the LOD/MDD for any given test, 90% of the LOD/MDD for that test was used in the analysis (Papa L et al., 2012).

### Commercial ELISA Kits

The following markers were detected using commercially available ELISA kits, as per manufacturer's instructions: 8OHdG (Cell Biolabs); BTA (Polymedco); CK18 (IDL); Clusterin (R&D Systems; Quantikine ELISA Human Clusterin, DCLU00), Creatinine (Randox Rx Daytona); CXCL16 (R&D Systems); Cystatin B (R&D Systems); Cystatin C (Randox Daytona Rx); FAS (RayBio); HAD (MyBioSource); Microalbumin (Randox Rx Daytona); Midkine (CellMid); MMP9NGAL (R&D Systems; Quantikine ELISA Human MMP-9/NGAL Complex); MMP9TIMP1 (R&D Systems); PAI-1/Tpa (AssayPro); Progranulin (R&D Systems); TUP (Bradford Assay A595nm); TGFB1 (R&D Systems); Thrombomodulin (R&D Systems) and TPA (Abcam).

### Infection

Infection was a clinical-based diagnosis based on the following: patient clinical history, biomarkers and dipstick analysis. Infection may also be determined using UTI multiplex array (e.g. Randox Urinary Track Multiplex Assay) which involves extracting DNA from urine samples followed by an amplification (a single tube 28-plex PCR reaction), hybridisation and detection.

### Creatinine, Osmolality and TUP

Creatinine (μmol/L) measurements were determined using a quantitative in vitro diagnostic kit from Randox Laboratories (Catalogue No CR3814), and the results were collected from a Daytona RX Series Clinical Analyser (Randox Laboratories Ltd). The creatinine assay is linear up to 66000 μmol/L and has a sensitivity of 310 μmol/L.

Osmolality (mOsm) was determined using a Löser Micro-Osmometer (Type 15) (Löser Messtechnik, Berlin, Germany). Briefly, the Osmometer was calibrated using three independent readings of distilled water (0.1 ml) and a 300-mOsm standard supplied with the instrument. Calibration was confirmed by measuring the mOsm of a freshly prepared 0.9% NaCl solution (mean 286±3 mOsm, n=3). Instrument calibration was also verified at the end of analysis using the same 0.9% NaCl solution (mean 280.3±0.58 mOsm, n=3) to check for drift.

Total urinary protein levels (mg/ml) were determined using a Bradford Assay Reagent Kit (A₅₉₅ nm) (Pierce, Rockford, Ill., USA) and BSA as standard (1 mg/ml). Patient samples (10 μl/patient) were mixed with Bradford Reagent (1 ml) and read on a Hitachi Spectrophotometer (Model No U-2800) at A₅₉₅ nm. The levels in the urine samples were determined from a BSA calibration chart (0-5 mg/ml, n=3).

### Statistical Analysis

Statistical analyses were undertaken using the Mann-Whitney U test (IBM SPSS v25) and R (Wilcoxon) to identify markers which were differentially expressed between control and bladder cancer.

Markers which contributed to algorithms were identified by binary logistic regression (Forward and Backward Wald) using SPSS and R (stats, glmnet (Lasso), glmulti).

Statistical significance was taken at the p<0.05 level.

Examples are shown below (SPSS Analyses (HaBio Females) for 4 biomarker combinations; and 4 biomarkers+infection).

SPSS Analysis (HaBio) - Females (4
Biomarkers) Classification Table^{a}

| Predicted | | |
|---|---|---|
| | All cancers | Percentage |

| Observed | | 0 | 1 | Correct |
|---|---|---|---|---|
| All | 0 | 116 | 22 | 84.1 |
| cancers | 1 | 13 | 36 | 73.5 |

| | |
|---|---|
| Overall Percentage | 81.3 |

^{a}The cut-off value is .250
0 = no cancer,
1 = cancer present

### Variables in the Equation

| | | B | S.E. | Wald | df | Sig. | Exp(B) |
|---|---|---|---|---|---|---|---|
| Step 1^{a} | BTA | .032 | .010 | 11.302 | 1 | .001 | 1.033 |
| | IL12p70 | −.850 | .236 | 12.949 | 1 | .000 | .427 |
| | IL13 | .390 | .101 | 15.016 | 1 | .000 | 1.477 |
| | Midkine | .001 | .000 | 6.803 | 1 | .009 | 1.001 |
| | Constant | −1.839 | .805 | 5.218 | 1 | .022 | .159 |

^{a}Variable(s) entered on step 1: BTA, IL12p70, IL13, Midkine.

### Area Under the Curve

Test Result Variable(s): Predicted probability

| | | | Asymptotic 95% Confidence Interval | |
|---|---|---|---|---|
| | Std. | Asymptotic | Lower | Upper |
| AUC | Error^{a} | Sig.^{b} | Bound | Bound |
| 0.867 | 0.030 | 0.000 | 0.807 | .927 |

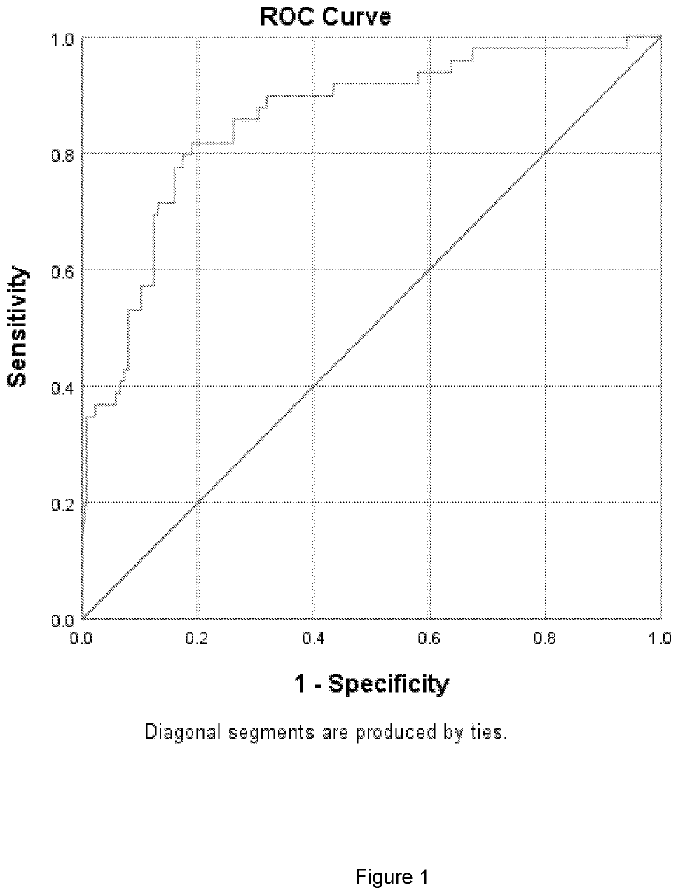

^{a}Under the nonparametric assumption
^{b}Null hypothesis: true area = 0.5
The calculated ROC curve is shown in FIG. 1.

SPSS Analysis (HaBio) - Females (4 Biomarkers +
Infection) Classification Table^{a}

| Predicted | | |
|---|---|---|
| | All cancers | Percentage |

| Observed | | 0 | 1 | Correct |
|---|---|---|---|---|
| All | 0 | 119 | 19 | 86.2 |
| cancers | 1 | 7 | 42 | 85.7 |

| | |
|---|---|
| Overall Percentage | 86.1 |

^{a}The cut-off value is .250
0 = no cancer,
1 = cancer present

### Variables in the Equation

| | | B | S.E. | Wald | df | Sig. | Exp(B) |
|---|---|---|---|---|---|---|---|
| Step 1^{a} | BTA | .034 | .012 | 8.694 | 1 | .003 | 1.035 |
| | IL12p70 | −.769 | .259 | 8.792 | 1 | .003 | .463 |
| | IL13 | .377 | .115 | 10.665 | 1 | .001 | 1.457 |
| | Midkine | .002 | .001 | 10.048 | 1 | .002 | 1.002 |
| | Infection (1) | 3.941 | 1.117 | 12.452 | 1 | .000 | 51.448 |
| | Constant | −5.374 | 1.435 | 14.029 | 1 | .000 | .005 |

^{a}Variable(s) entered on step 1: BTA, IL12p70, IL13, Midkine, Infection.

### Area Under the Curve

Test Result Variable(s): Predicted probability

| | | | Asymptotic 95% Confidence Interval | |
|---|---|---|---|---|
| | Std. | Asymptotic | Lower | Upper |
| AUC | Error^{a} | Sig.^{b} | Bound | Bound |
| 0.923 | 0.021 | 0.000 | 0.882 | 0.963 |

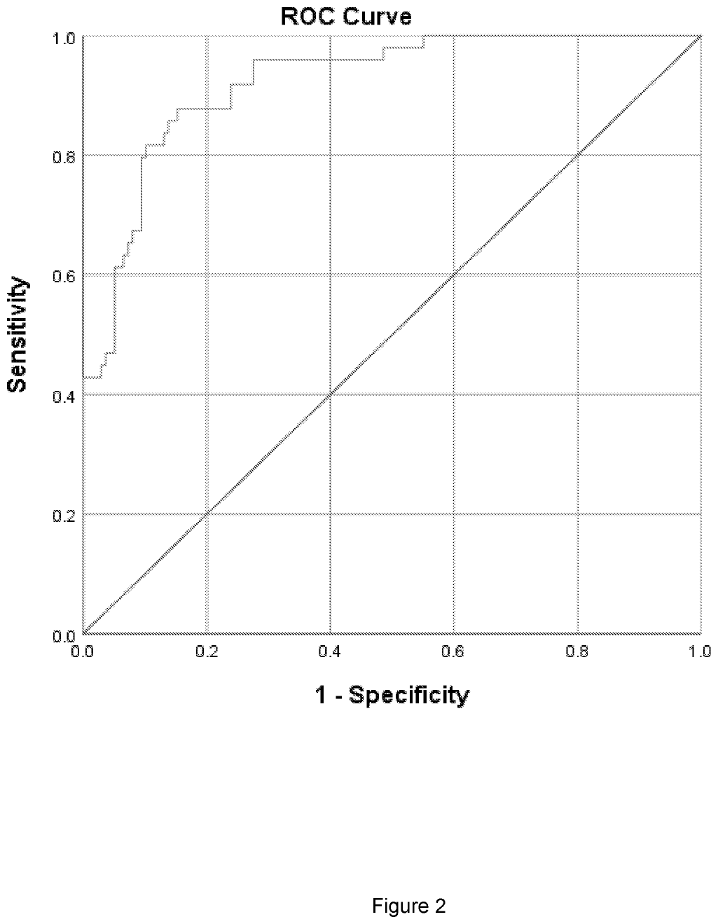

^{a}Under the nonparametric assumption
^{b}Null hypothesis: true area = 0.5
The calculated ROC curve is shown in FIG. 2.

### Incorporating Infection Status

Incorporating an initial infection test increases the AUCs and reduces the number of markers needed to diagnose bladder cancer.

| | AUC not incorporating | AUC incorporating |
|---|---|---|
| Biomarkers | infection status | infection status |
| IL-13 | 0.686 | 0.830 |
| IL12p70 | 0.632 | 0.796 |
| BTA | 0.754 | 0.868 |
| Midkine | 0.694 | 0.863 |
| IL-13 + IL12p70 | 0.783 | 0.869 |
| IL-13 + BTA | 0.830 | 0.902 |
| IL-13 + Midkine | 0.782 | 0.895 |
| IL12p70 + BTA | 0.812 | 0.885 |
| IL12p70 + Midkine | 0.748 | 0.877 |
| BTA + Midkine | 0.753 | 0.882 |

### Results and Discussion

Certain biomarkers were significantly higher in female bladder cancer patients, including BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF and Triglycerides 1 (p<0.050; Mann Whitney test). The following biomarkers were the most significant: BTA, Midkine, PAI-1/tPA, Clusterin, IL-8, Microalbumin, MMP9NGAL, NSE, Cystatin C, d-Dimer, IL-7.

The algorithms identified by the inventors are surprising and the biomarkers included in the algorithms could not have been predicted.

Table 1 shows the hypothesis test summary using the Mann-Whitney U test. When a biomarker had a correlation of greater than, or equal to 0.7, this biomarker could be substituted with a biomarker that it correlates with, as the two biomarkers are related. Significance values of less than 0.7 shows that the two biomarkers are independent.

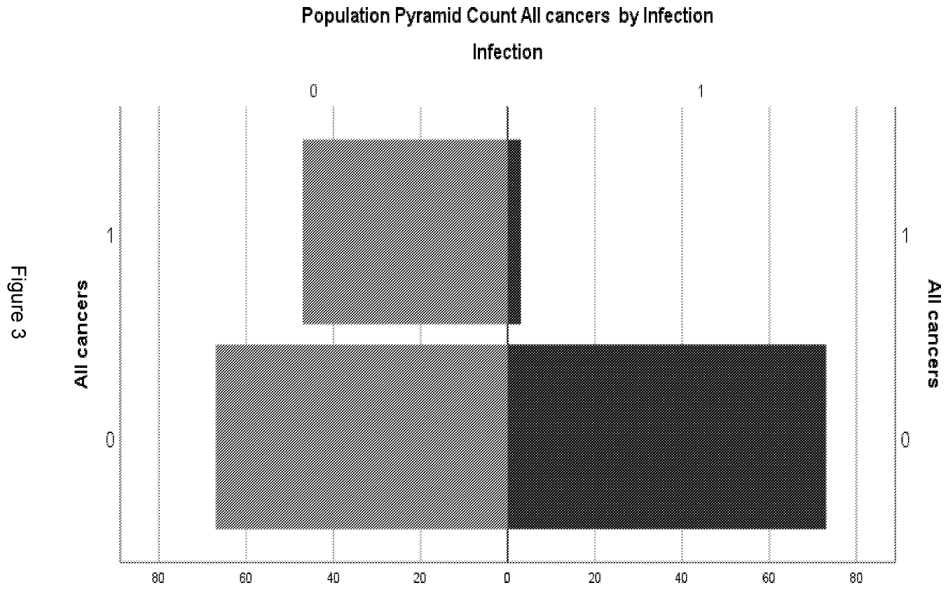

FIG. 3 shows that female patients presenting with haematuria are likely to have haematuria because of infection (bacterial and/or viral) and not because of cancer. The right half of the diagram represents patients with infection, the left half patients without infection; and the lower half of the diagram patients without cancer, the upper half patients with cancer; hence, approximately 2 of the 76 patients with infection have cancer. As it is desirable to rule out bladder cancer and avoid cystoscopy, testing haematuric females presenting at the GP's surgery for infection (using any methodology/test) enables infection positive females to be sent home with a course of antibiotics thus easing the NHS referral burden. Based on FIG. 3, approximately 76 out of 184 patients could be sent home and only 2 incorrectly (the 2 missed patients would be sent for referral on failure of the antibiotics). The remaining ˜108 patients without infection would be subject to the biomarker test.

The following are a list of abbreviations used in the present specification:
80HdG OxiSelect Oxidative DNA Damage
ACR Albumin:Creatinine Ratio
AUC Area Under Curve
BLC Blue Light Cystoscopy
BMI Body Mass Index
BTA Bladder Tumour Antigen
CEA Carcinoembryonic Antigen
CIS Carcinoma in situ
CK-18 Cytokeratin 18
CKD Chronic kidney disease
CRP C Reactive Protein
CRS Clinical Risk Score
EGF Epidermal Growth Factor
FAS FAS Protein
FDP Fibrinogen Degradation Products
FINJEM Finnish Job Exposure Matrix
GP General Practitioner
HRP Horse Radish Peroxidase
IL-2 Interleukin 2
IL-3 Interleukin 3
IL-4 Interleukin 4
IL-6 Interleukin 6
IL-7 Interleukin 7
IL-8 Interleukin 8
IL-10 Interleukin 10
IL-12p70 Interleukin 12p70
IL-13 Interleukin 13
IL-18 Interleukin 18
IL-23 Interleukin 23
LOD Limit of Detection
MCP Monocyte Chemotactic Protein
MDD Mean Detectable Dose
MMP9 Matrix Metalloprotein 9
MMP-9/NGALMatrix Metalloprotein 9/Neutrophil Gelatinase Associated Lipocalin Complex
MMP9/TIMP1 Matrix Metalloprotein 9/Tissue Inhibitor of Metalloprotein 1
NGAL Neutrophil Gelatinase Associated Lipocalin
NICE National Institute for Clinical Excellence
NMP22 Nuclear Matrix Protein 22
NSE Neuron Specific Enolase
NVH Non-Visible Haematuria
ONS Office for National Statistics
PAI-1/tPA Plasminogen Activator Inhibitor 1/Tissue Plasminogen Activator
POC Point of Care
ROC Receiver Operating Curve
SD Standard Deviation
SDS-PAGE Sodium Dodecyl Sulphate-Polyacrylamide Gel Electrophoresis
sTNFR1 Soluble Tumour Necrosis Factor 1
sTNFR2 Soluble Tumour Necrosis Factor 2
TGFB1 Transforming Growth Factor Beta 1
TM Thrombomodulin
TPA Tissue Type Plasminogen Activator
TPSA Total Prostate Specific Antigen
TUP Total Urinary Protein
UTI Urinary Tract Infection
VEGF Vascular Endothelial Growth Factor
WLC White Light Cystoscopy
   Table 1 shows the hypothesis test summary using the Mann-Whitney U test

| Biomarker | Matrix | Significance |
|---|---|---|
| 80HdG | Urine | 0.019 |
| ACR | Urine | 0.000 |
| BTA | Urine | 0.000 |
| CD44 | Serum | 0.094 |
| CEA | Serum | 0.037 |
| CK18 | Urine | 0.000 |
| CK20 | Urine | 0.299 |
| Clusterin | Urine | 0.000 |
| Creatinine | Urine | 0.002 |
| Creatinine μmolL | Urine | 0.002 |
| CRP | Urine | 0.071 |
| CRP | Serum | 0.338 |
| CXCL16 | Urine | 0.000 |
| Cystatin B | Urine | 0.002 |
| Cystatin C | Urine | 0.000 |
| Cystatin C | Serum | 0.820 |
| d-Dimer | Urine | 0.000 |
| EGF | Urine | 0.000 |
| EGF | Serum | 0.000 |
| FABP-A | Serum | 0.726 |
| FAS | Urine | 0.016 |
| GRO | Serum | 0.094 |
| HAD | Urine | 0.002 |
| IFN gamma | Urine | 0.124 |
| IFN gamma | Serum | 0.416 |
| IL-1a | Urine | 0.000 |
| IL-1a | Serum | 0.043 |
| IL-1b | Urine | 0.000 |
| IL-1b | Serum | 0.551 |
| IL-2 | Urine | 0.756 |
| IL-2 | Serum | 0.777 |
| IL-3 | Urine | 0.396 |
| IL-4 | Urine | 0.019 |
| IL-4 | Serum | 0.613 |
| IL-6 | Urine | 0.001 |
| IL-6 | Serum | 0.075 |
| IL-7 | Urine | 0.000 |
| IL-8 | Urine | 0.000 |
| IL-8 | Serum | 0.238 |
| IL-10 | Urine | 0.087 |
| IL-10 | Serum | 0.110 |
| IL12p70 | Urine | 0.002 |
| IL-13 | Urine | 0.000 |
| IL-18 | Serum | 0.134 |
| IL-23 | Urine | 0.801 |
| LASP-1 | Serum | 0.925 |
| M30 | Serum | 0.903 |
| M2PK | Serum | 0.566 |
| MCP-1 | Urine | 0.000 |
| MCP-1 | Serum | 0.190 |
| Microalbumin | Urine | 0.000 |
| Midkine | Urine | 0.000 |
| MMP9 | Urine | 0.293 |
| MMP9NGAL | Urine | 0.000 |
| MMP9TIMP1 | Urine | 0.001 |
| NGAL | Urine | 0.002 |
| NSE | Urine | 0.000 |
| Osmolality | Urine | 0.0086 |
| PAI-1/tPA | Serum | 0.000 |
| pERK | Urine | 0.164 |
| Progranulin | Urine | 0.002 |
| Prolactin | Serum | 0.640 |
| TUP | Urine | 0.000 |
| PSA-TPSA | Serum | 0.297 |
| S100A4 | Serum | 0.579 |
| sIL-2Ra | Urine | 0.516 |
| SIL-6SR | Urine | 0.463 |
| TGFB1 | Urine | 0.001 |
| Thrombomodulin | Urine | 0.000 |
| TNFa | Urine | 0.101 |
| TNFa | Serum | 0.347 |
| sTNFRI | Urine | 0.000 |
| sTNFRII | Urine | 0.156 |
| TPA | Urine | 0.010 |
| VEGF | Urine | 0.000 |
| VEGF | Serum | 0.101 |
| HDL | Serum | 0.549 |
| LDL | Serum | 0.082 |
| Triglycerides | Serum | 0.008 |
| Cholesterol | Serum | 0.521 |

Table 2 shows the AUC, sensitivities and specificities for biomarker combinations generated using GLMulti (using R), after forward and backward Wald binary logistic regression. “u” means the biomarker is in urinary form and “s” means the biomarker is in serum form.

| Marker | | | | | | | | |
|---|---|---|---|---|---|---|---|---|
| Combination | Correct | Total | Correct | Total | Best | | | |
| Algorithms | Controls | Controls | UC | UC | Threshold | Sensitivity | Specificity | AUC |
| u_BTA + u_Cystatin_B + | 118 | 135 | 38 | 47 | 0.294 | 0.809 | 0.874 | 0.894 |
| u_EGF + s_EGF + | | | | | | | | |
| u_IL12p70 + u_IL13 + | | | | | | | | |
| u_Midkine + s_PAI_1tPA | | | | | | | | |
| u_BTA + u_Clusterin + | 118 | 135 | 38 | 47 | 0.290 | 0.809 | 0.874 | 0.893 |
| u_Cystatin_B + u_EGF + | | | | | | | | |
| s_EGF + u_IL12p70 + | | | | | | | | |
| u_IL13 + u_Midkine + | | | | | | | | |
| s_PAI_1tPA | | | | | | | | |
| u_BTA + u_Cystatin_B + | 118 | 131 | 37 | 46 | 0.342 | 0.804 | 0.901 | 0.913 |
| u_EGF + s_EGF + | | | | | | | | |
| u_IL12p70 + u_IL13 + | | | | | | | | |
| u_Midkine + s_PAI_1tPA + | | | | | | | | |
| s_Triglycerides | | | | | | | | |
| u_BTA + u_Cystatin_B + | 114 | 135 | 39 | 47 | 0.270 | 0.830 | 0.844 | 0.897 |
| u_EGF + s_EGF + | | | | | | | | |
| u_IL8 + u_IL12p70 + | | | | | | | | |
| u_IL13 + u_Midkine + | | | | | | | | |
| s_PAI_1tPA | | | | | | | | |
| u_BTA + u_Clusterin + | 111 | 135 | 39 | 47 | 0.233 | 0.830 | 0.822 | 0.892 |
| u_Cystatin_B + s_EGF + | | | | | | | | |
| u_IL12p70 + u_IL13 + | | | | | | | | |
| u_Midkine + s_PAI_1tPA | | | | | | | | |
| u_BTA + u_Clusterin + | 115 | 131 | 37 | 46 | 0.331 | 0.804 | 0.878 | 0.916 |
| u_Cystatin_B + u_EGF + | | | | | | | | |
| s_EGF + u_IL12p70 + | | | | | | | | |
| u_IL13 + u_Midkine + | | | | | | | | |
| s_PAI_1tPA + | | | | | | | | |
| s_Triglycerides | | | | | | | | |
| u_BTA + u_Clusterin + | 115 | 131 | 36 | 46 | 0.356 | 0.783 | 0.878 | 0.911 |
| u_Cystatin_B + s_EGF + | | | | | | | | |
| u_IL12p70 + u_IL13 + | | | | | | | | |
| u_Midkine + s_PAI_1tPA + | | | | | | | | |
| s_Triglycerides | | | | | | | | |
| u_BTA + u_Cystatin_B + | 109 | 136 | 41 | 48 | 0.184 | 0.854 | 0.801 | 0.896 |
| u_EGF + u_IL12p70 + | | | | | | | | |
| u_IL13 + u_Midkine + | | | | | | | | |
| s_PAI_1tPA | | | | | | | | |
| u_BTA + u_Clusterin + | 109 | 136 | 41 | 48 | 0.184 | 0.854 | 0.801 | 0.896 |
| u_Cystatin_B + u_EGF + | | | | | | | | |
| u_IL12p70 + u_IL13 + | | | | | | | | |
| u_Midkine + s_PAI_1tPA | | | | | | | | |
| u_BTA + u_Clusterin + | 113 | 135 | 39 | 47 | 0.274 | 0.830 | 0.837 | 0.896 |
| u_Cystatin_B + u_EGF + | | | | | | | | |
| s_EGF + u_IL8 + | | | | | | | | |
| u_IL12p70 + u_IL13 + | | | | | | | | |
| u_Midkine + s_PAI_1tPA | | | | | | | | |
| u_BTA + u_Cystatin_B + | 120 | 131 | 37 | 46 | 0.349 | 0.804 | 0.916 | 0.920 |
| u_EGF + s_EGF + | | | | | | | | |
| u_IL8 + u_IL12p70 + | | | | | | | | |
| u_IL13 + u_Midkine + | | | | | | | | |
| s_PAI_1tPA + | | | | | | | | |
| s_Triglycerides | | | | | | | | |
| u_BTA + u_Clusterin + | 108 | 131 | 38 | 46 | 0.255 | 0.826 | 0.824 | 0.902 |
| s_EGF + u_IL12p70 + | | | | | | | | |
| u_IL13 + u_Midkine + | | | | | | | | |
| s_PAI_1tPA + | | | | | | | | |
| s_Triglycerides | | | | | | | | |
| u_BTA + u_IL12p70 + | 109 | 136 | 40 | 48 | 0.198 | 0.833 | 0.801 | 0.884 |
| u_IL13 + u_Midkine + | | | | | | | | |
| s_PAI_1tPA | | | | | | | | |
| u_BTA + u_IL12p70 + | 108 | 136 | 40 | 48 | 0.201 | 0.833 | 0.794 | 0.865 |
| u_IL13 + u_Midkine | | | | | | | | |
| u_BTA + u_IL12p70 + | 108 | 136 | 40 | 48 | 0.195 | 0.833 | 0.794 | 0.884 |
| u_IL13 + u_Midkine + | | | | | | | | |
| s_PAI_1tPA + u_Clusterin | | | | | | | | |
| u_BTA + u_IL12p70 + | 112 | 136 | 39 | 48 | 0.231 | 0.813 | 0.824 | 0.889 |
| u_IL13 + u_Midkine + | | | | | | | | |
| s_PAI_1tPA + | | | | | | | | |
| u_Cystatin_B | | | | | | | | |
| u_BTA + u_IL12p70 + | 116 | 136 | 37 | 48 | 0.299 | 0.771 | 0.853 | 0.882 |
| u_IL13 + u_Midkine + | | | | | | | | |
| s_PAI_1tPA + u_EGF | | | | | | | | |
| u_BTA + u_IL12p70 + | 112 | 135 | 38 | 47 | 0.258 | 0.809 | 0.830 | 0.884 |
| u_IL13 + u_Midkine + | | | | | | | | |
| s_PAI_1tPA + s_EGF | | | | | | | | |
| u_BTA + u_IL12p70 + | 102 | 131 | 39 | 46 | 0.182 | 0.848 | 0.779 | 0.892 |
| u_IL13 + u_Midkine + | | | | | | | | |
| s_PAI_1tPA + | | | | | | | | |
| s_Triglycerides | | | | | | | | |
| u_BTA + u_IL12p70 + | 111 | 137 | 42 | 50 | 0.213 | 0.840 | 0.810 | 0.870 |
| u_IL13 + u_Midkine + | | | | | | | | |
| u_Clusterin | | | | | | | | |
| u_BTA + u_IL12p70 + | 111 | 137 | 42 | 50 | 0.194 | 0.840 | 0.810 | 0.878 |
| u_IL13 + u_Midkine + | | | | | | | | |
| u_Cystatin_B | | | | | | | | |
| u_BTA + u_IL12p70 + | 108 | 137 | 42 | 50 | 0.192 | 0.840 | 0.788 | 0.865 |
| u_IL13 + u_Midkine + | | | | | | | | |
| u_EGF | | | | | | | | |
| u_BTA + u_IL12p70 + | 102 | 135 | 40 | 47 | 0.200 | 0.851 | 0.756 | 0.877 |
| u_IL13 + u_Midkine + | | | | | | | | |
| s_EGF | | | | | | | | |
| u_BTA + u_IL12p70 + | 105 | 131 | 36 | 46 | 0.203 | 0.783 | 0.802 | 0.864 |
| u_IL13 + u_Midkine + | | | | | | | | |
| s_Triglycerides | | | | | | | | |
| u_BTA + u_IL12p70 + | 104 | 136 | 40 | 48 | 0.181 | 0.833 | 0.765 | 0.869 |
| u_IL13 + u_IL7 + | | | | | | | | |
| s_PAI_1tPA | | | | | | | | |
| u_BTA + u_IL12p70 + | 103 | 136 | 40 | 48 | 0.182 | 0.833 | 0.757 | 0.869 |
| u_IL13 + u_sTNFRI + | | | | | | | | |
| s_PAI_1tPA | | | | | | | | |
| u_BTA + u_IL12p70 + | 104 | 136 | 40 | 47 | 0.180 | 0.851 | 0.765 | 0.863 |
| u_IL13 + u_Cystatin_C + | | | | | | | | |
| s_PAI_1tPA | | | | | | | | |
| u_CK18 + u_IL12p70 + | 111 | 134 | 36 | 44 | 0.216 | 0.818 | 0.828 | 0.862 |
| u_IL13 + u_Midkine + | | | | | | | | |
| s_PAI_1tPA | | | | | | | | |
| u_CK18 + u_IL12p70 + | 115 | 134 | 33 | 44 | 0.271 | 0.750 | 0.858 | 0.850 |
| u_IL13 + u_IL7 + | | | | | | | | |
| s_PAI_1tPA | | | | | | | | |
| u_CK18 + u_IL12p70 + | 97 | 135 | 38 | 46 | 0.188 | 0.826 | 0.719 | 0.833 |
| u_IL13 + u_IL7 | | | | | | | | |
| u_BTA + u_IL12p70 + | 115 | 136 | 35 | 46 | 0.262 | 0.761 | 0.846 | 0.879 |
| u_IL13 + u_Midkine + | | | | | | | | |
| s_PAI_1tPA + u_HdG80 | | | | | | | | |
| u_BTA + u_IL12p70 + | 109 | 137 | 40 | 48 | 0.205 | 0.833 | 0.796 | 0.864 |
| u_IL13 + u_Midkine + | | | | | | | | |
| u_HdG80 | | | | | | | | |
| u_BTA + u_IL12p70 + | 106 | 136 | 41 | 48 | 0.190 | 0.854 | 0.779 | 0.883 |
| u_IL13 + u_Midkine + | | | | | | | | |
| s_PAI_1tPA + u_ACR | | | | | | | | |
| u_BTA + u_IL12p70 + | 110 | 137 | 41 | 50 | 0.212 | 0.820 | 0.803 | 0.867 |
| u_IL13 + u_Midkine + | | | | | | | | |
| u_ACR | | | | | | | | |
| u_BTA + u_IL12p70 + | 110 | 136 | 40 | 48 | 0.206 | 0.833 | 0.809 | 0.882 |
| u_IL13 + u_Midkine + | | | | | | | | |
| s_PAI_1tPA + s_CEA | | | | | | | | |
| u_BTA + u_IL12p70 + | 108 | 136 | 41 | 48 | 0.209 | 0.854 | 0.794 | 0.868 |
| u_IL13 + u_Midkine + | | | | | | | | |
| s_CEA | | | | | | | | |
| u_BTA + u_IL12p70 + | 108 | 134 | 36 | 44 | 0.202 | 0.818 | 0.806 | 0.875 |
| u_IL13 + u_Midkine + | | | | | | | | |
| s_PAI_1tPA + u_CK18 | | | | | | | | |
| u_BTA + u_IL12p70 + | 110 | 135 | 38 | 46 | 0.212 | 0.826 | 0.815 | 0.858 |
| u_IL13 + u_Midkine + | | | | | | | | |
| u_CK18 | | | | | | | | |
| u_BTA + u_IL12p70 + | 111 | 136 | 38 | 48 | 0.221 | 0.792 | 0.816 | 0.879 |
| u_IL13 + u_Midkine + | | | | | | | | |
| s_PAI_1tPA + | | | | | | | | |
| u_Creatinine | | | | | | | | |
| u_BTA + u_IL12p70 + | 109 | 137 | 42 | 50 | 0.205 | 0.840 | 0.796 | 0.866 |
| u_IL13 + u_Midkine + | | | | | | | | |
| u_Creatinine | | | | | | | | |
| u_BTA + u_IL12p70 + | 111 | 136 | 38 | 48 | 0.221 | 0.792 | 0.816 | 0.879 |
| u_IL13 + u_Midkine + | | | | | | | | |
| s_PAI_1tPA + | | | | | | | | |
| u_Creatinine_umolL | | | | | | | | |
| u_BTA + u_IL12p70 + | 109 | 137 | 42 | 50 | 0.205 | 0.840 | 0.796 | 0.866 |
| u_IL13 + u_Midkine + | | | | | | | | |
| u_Creatinine_umolL | | | | | | | | |
| u_BTA + u_IL12p70 + | 106 | 135 | 39 | 46 | 0.188 | 0.848 | 0.785 | 0.877 |
| u_IL13 + u_Midkine + | | | | | | | | |
| s_PAI_1tPA + u_CXCL16 | | | | | | | | |
| u_BTA + u_IL12p70 + | 109 | 136 | 40 | 48 | 0.204 | 0.833 | 0.801 | 0.865 |
| u_IL13 + u_Midkine + | | | | | | | | |
| u_CXCL16 | | | | | | | | |
| u_BTA + u_IL12p70 + | 107 | 136 | 40 | 47 | 0.191 | 0.851 | 0.787 | 0.882 |
| u_IL13 + u_Midkine + | | | | | | | | |
| s_PAI_1tPA + | | | | | | | | |
| u_Cystatin_C | | | | | | | | |
| u_BTA + u_IL12p70 + | 104 | 137 | 43 | 49 | 0.171 | 0.878 | 0.759 | 0.869 |
| u_IL13 + u_Midkine + | | | | | | | | |
| u_Cystatin_C | | | | | | | | |
| u_BTA + u_IL12p70 + | 106 | 136 | 41 | 48 | 0.190 | 0.854 | 0.779 | 0.881 |
| u_IL13 + u_Midkine + | | | | | | | | |
| s_PAI_1tPA + u_dDimer | | | | | | | | |
| u_BTA + u_IL12p70 + | 110 | 137 | 42 | 50 | 0.205 | 0.840 | 0.803 | 0.866 |
| u_IL13 + u_Midkine + | | | | | | | | |
| u_dDimer | | | | | | | | |
| u_BTA + u_IL12p70 + | 108 | 136 | 40 | 48 | 0.198 | 0.833 | 0.794 | 0.883 |
| u_IL13 + u_Midkine + | | | | | | | | |
| s_PAI_1tPA + u_FAS | | | | | | | | |
| u_BTA + u_IL12p70 + | 109 | 137 | 42 | 50 | 0.207 | 0.840 | 0.796 | 0.870 |
| u_IL13 + u_Midkine + | | | | | | | | |
| u_FAS | | | | | | | | |
| u_BTA + u_IL12p70 + | 107 | 136 | 40 | 47 | 0.189 | 0.851 | 0.787 | 0.880 |
| u_IL13 + u_Midkine + | | | | | | | | |
| s_PAI_1tPA + u_HAD | | | | | | | | |
| u_BTA + u_IL12p70 + | 110 | 137 | 41 | 49 | 0.207 | 0.837 | 0.803 | 0.863 |
| u_IL13 + u_Midkine + | | | | | | | | |
| u_HAD | | | | | | | | |
| u_BTA + u_IL12p70 + | 109 | 136 | 40 | 48 | 0.199 | 0.833 | 0.801 | 0.883 |
| u_IL13 + u_Midkine + | | | | | | | | |
| s_PAI_1tPA + u_IL1a | | | | | | | | |
| u_BTA + u_IL12p70 + | 110 | 137 | 42 | 50 | 0.208 | 0.840 | 0.803 | 0.867 |
| u_IL13 + u_Midkine + | | | | | | | | |
| u_IL1a | | | | | | | | |
| u_BTA + u_IL12p70 + | 110 | 135 | 38 | 47 | 0.206 | 0.809 | 0.815 | 0.882 |
| u_IL13 + u_Midkine + | | | | | | | | |
| s_PAI_1tPA + s_IL1a | | | | | | | | |
| u_BTA + u_IL12p70 + | 107 | 135 | 39 | 47 | 0.204 | 0.830 | 0.793 | 0.862 |
| u_IL13 + u_Midkine + | | | | | | | | |
| s_IL1a | | | | | | | | |
| u_BTA + u_IL12p70 + | 110 | 136 | 39 | 48 | 0.201 | 0.813 | 0.809 | 0.883 |
| u_IL13 + u_Midkine + | | | | | | | | |
| s_PAI_1tPA + u_IL1b | | | | | | | | |
| u_BTA + u_IL12p70 + | 110 | 137 | 42 | 50 | 0.208 | 0.840 | 0.803 | 0.867 |
| u_IL13 + u_Midkine + | | | | | | | | |
| u_IL1b | | | | | | | | |
| u_BTA + u_IL12p70 + | 106 | 136 | 43 | 48 | 0.180 | 0.896 | 0.779 | 0.883 |
| u_IL13 + u_Midkine + | | | | | | | | |
| s_PAI_1tPA + u_IL4 | | | | | | | | |
| u_BTA + u_IL12p70 + | 105 | 137 | 44 | 50 | 0.181 | 0.880 | 0.766 | 0.865 |
| u_IL13 + u_Midkine + | | | | | | | | |
| u_IL4 | | | | | | | | |
| u_BTA + u_IL12p70 + | 110 | 136 | 40 | 48 | 0.199 | 0.833 | 0.809 | 0.883 |
| u_IL13 + u_Midkine + | | | | | | | | |
| s_PAI_1tPA + u_IL6 | | | | | | | | |
| u_BTA + u_IL12p70 + | 108 | 137 | 42 | 50 | 0.184 | 0.840 | 0.788 | 0.868 |
| u_IL13 + u_Midkine + | | | | | | | | |
| u_IL6 | | | | | | | | |
| u_BTA + u_IL12p70 + | 108 | 136 | 40 | 48 | 0.187 | 0.833 | 0.794 | 0.882 |
| u_IL13 + u_Midkine + | | | | | | | | |
| s_PAI_1tPA + u_IL7 | | | | | | | | |
| u_BTA + u_IL12p70 + | 109 | 137 | 42 | 50 | 0.203 | 0.840 | 0.796 | 0.866 |
| u_IL13 + u_Midkine + | | | | | | | | |
| u_IL7 | | | | | | | | |
| u_BTA + u_IL12p70 + | 106 | 136 | 41 | 48 | 0.187 | 0.854 | 0.779 | 0.883 |
| u_IL13 + u_Midkine + | | | | | | | | |
| s_PAI_1tPA + u_IL8 | | | | | | | | |
| u_BTA + u_IL12p70 + | 110 | 137 | 42 | 50 | 0.208 | 0.840 | 0.803 | 0.867 |
| u_IL13 + u_Midkine + | | | | | | | | |
| u_IL8 | | | | | | | | |
| u_BTA + u_IL12p70 + | 110 | 136 | 39 | 48 | 0.206 | 0.813 | 0.809 | 0.883 |
| u_IL13 + u_Midkine + | | | | | | | | |
| s_PAI_1tPA + u_MCP_1 | | | | | | | | |
| u_BTA + u_IL12p70 + | 108 | 137 | 43 | 50 | 0.177 | 0.860 | 0.788 | 0.870 |
| u_IL13 + u_Midkine + | | | | | | | | |
| u_MCP_1 | | | | | | | | |
| u_BTA + u_IL12p70 + | 110 | 136 | 40 | 48 | 0.198 | 0.833 | 0.809 | 0.884 |
| u_IL13 + u_Midkine + | | | | | | | | |
| s_PAI_1tPA + | | | | | | | | |
| u_Microalbumin | | | | | | | | |
| u_BTA + u_IL12p70 + | 110 | 137 | 42 | 50 | 0.208 | 0.840 | 0.803 | 0.867 |
| u_IL13 + u_Midkine + | | | | | | | | |
| u_Microalbumin | | | | | | | | |
| u_BTA + u_IL12p70 + | 110 | 136 | 40 | 48 | 0.197 | 0.833 | 0.809 | 0.883 |
| u_IL13 + u_Midkine + | | | | | | | | |
| s_PAI_1tPA + | | | | | | | | |
| u_MMP9NGAL | | | | | | | | |
| u_BTA + u_IL12p70 + | 112 | 137 | 42 | 50 | 0.206 | 0.840 | 0.818 | 0.870 |
| u_IL13 + u_Midkine + | | | | | | | | |
| u_MMP9NGAL | | | | | | | | |
| u_BTA + u_IL12p70 + | 107 | 136 | 40 | 48 | 0.190 | 0.833 | 0.787 | 0.882 |
| u_IL13 + u_Midkine + | | | | | | | | |
| s_PAI_1tPA + | | | | | | | | |
| u_MMP9TIMP1 | | | | | | | | |
| u_BTA + u_IL12p70 + | 108 | 137 | 42 | 50 | 0.203 | 0.840 | 0.788 | 0.866 |
| u_IL13 + u_Midkine + | | | | | | | | |
| u_MMP9TIMP1 | | | | | | | | |
| u_BTA + u_IL12p70 + | 109 | 136 | 41 | 48 | 0.190 | 0.854 | 0.801 | 0.887 |
| u_IL13 + u_Midkine + | | | | | | | | |
| s_PAI_1tPA + u_NGAL | | | | | | | | |
| u_BTA + u_IL12p70 + | 110 | 137 | 43 | 50 | 0.177 | 0.860 | 0.803 | 0.875 |
| u_IL13 + u_Midkine + | | | | | | | | |
| u_NGAL | | | | | | | | |
| u_BTA + u_IL12p70 + | 115 | 136 | 39 | 48 | 0.253 | 0.813 | 0.846 | 0.884 |
| u_IL13 + u_Midkine + | | | | | | | | |
| s_PAI_1tPA + u_NSE | | | | | | | | |
| u_BTA + u_IL12p70 + | 114 | 137 | 41 | 50 | 0.213 | 0.820 | 0.832 | 0.869 |
| u_IL13 + u_Midkine + | | | | | | | | |
| u_NSE | | | | | | | | |
| u_BTA + u_IL12p70 + | 110 | 136 | 39 | 47 | 0.195 | 0.830 | 0.809 | 0.880 |
| u_IL13 + u_Midkine + | | | | | | | | |
| s_PAI_1tPA + | | | | | | | | |
| u_Progranulin | | | | | | | | |
| u_BTA + u_IL12p70 + | 110 | 137 | 41 | 49 | 0.208 | 0.837 | 0.803 | 0.864 |
| u_IL13 + u_Midkine + | | | | | | | | |
| u_Progranulin | | | | | | | | |
| u_BTA + u_IL12p70 + | 108 | 136 | 40 | 48 | 0.198 | 0.833 | 0.794 | 0.883 |
| u_IL13 + u_Midkine + | | | | | | | | |
| s_PAI_1tPA + u_TUP | | | | | | | | |
| u_BTA + u_IL12p70 + | 114 | 137 | 41 | 50 | 0.213 | 0.820 | 0.832 | 0.870 |
| u_IL13 + u_Midkine + | | | | | | | | |
| u_TUP | | | | | | | | |
| u_BTA + u_IL12p70 + | 109 | 136 | 40 | 48 | 0.198 | 0.833 | 0.801 | 0.883 |
| u_IL13 + u_Midkine + | | | | | | | | |
| s_PAI_1tPA + u_TGFb1 | | | | | | | | |
| u_BTA + u_IL12p70 + | 108 | 137 | 43 | 50 | 0.178 | 0.860 | 0.788 | 0.868 |
| u_IL13 + u_Midkine + | | | | | | | | |
| u_TGFb1 | | | | | | | | |
| u_BTA + u_IL12p70 + | 111 | 136 | 38 | 48 | 0.223 | 0.792 | 0.816 | 0.880 |
| u_IL13 + u_Midkine + | | | | | | | | |
| s_PAI_1tPA + | | | | | | | | |
| u_Thrombomodulin | | | | | | | | |
| u_BTA + u_IL12p70 + | 109 | 137 | 42 | 50 | 0.203 | 0.840 | 0.796 | 0.866 |
| u_IL13 + u_Midkine + | | | | | | | | |
| u_Thrombomodulin | | | | | | | | |
| u_BTA + u_IL12p70 + | 107 | 136 | 41 | 48 | 0.188 | 0.854 | 0.787 | 0.885 |
| u_IL13 + u_Midkine + | | | | | | | | |
| s_PAI_1tPA + u_sTNFRI | | | | | | | | |
| u_BTA + u_IL12p70 + | 108 | 137 | 42 | 50 | 0.195 | 0.840 | 0.788 | 0.872 |
| u_IL13 + u_Midkine + | | | | | | | | |
| u_sTNFRI | | | | | | | | |
| u_BTA + u_IL12p70 + | 108 | 136 | 40 | 47 | 0.186 | 0.851 | 0.794 | 0.879 |
| u_IL13 + u_Midkine + | | | | | | | | |
| s_PAI_1tPA + u_TPA | | | | | | | | |
| u_BTA + u_IL12p70 + | 110 | 137 | 41 | 49 | 0.209 | 0.837 | 0.803 | 0.864 |
| u_IL13 + u_Midkine + | | | | | | | | |
| u_TPA | | | | | | | | |
| u_BTA + u_IL12p70 + | 112 | 136 | 38 | 48 | 0.225 | 0.792 | 0.824 | 0.881 |
| u_IL13 + u_Midkine + | | | | | | | | |
| s_PAI_1tPA + u_VEGF | | | | | | | | |
| u_BTA + u_IL12p70 + | 108 | 137 | 42 | 50 | 0.190 | 0.840 | 0.788 | 0.867 |
| u_IL13 + u_Midkine + | | | | | | | | |
| u_VEGF | | | | | | | | |
| u_BTA + | 111 | 137 | 39 | 50 | 0.213 | 0.780 | 0.810 | 0.848 |
| u_IL12p70_nom + | | | | | | | | |
| u_IL13_nom + | | | | | | | | |
| u_Midkine | | | | | | | | |
| u_BTA + | 111 | 137 | 39 | 50 | 0.213 | 0.780 | 0.810 | 0.848 |
| u_IL12p70_nom + | | | | | | | | |
| u_IL13_nom + | | | | | | | | |
| u_Midkine | | | | | | | | |
| u_CK18 + | 105 | 134 | 35 | 44 | 0.192 | 0.795 | 0.784 | 0.875 |
| u_IL12p70_nom + | | | | | | | | |
| u_IL13_nom + | | | | | | | | |
| u_Midkine + | | | | | | | | |
| s_PAI_1tPA | | | | | | | | |
| u_IL12p70 + u_IL13 + | 114 | 137 | 38 | 50 | 0.222 | 0.760 | 0.832 | 0.841 |
| u_Midkine | | | | | | | | |
| u_IL12p70 + u_IL13 + | 112 | 137 | 41 | 50 | 0.204 | 0.820 | 0.818 | 0.858 |
| u_BTA | | | | | | | | |
| u_IL12p70_nom + | 110 | 137 | 36 | 50 | 0.213 | 0.720 | 0.803 | 0.830 |
| u_IL13_nom + | | | | | | | | |
| u_Midkine | | | | | | | | |
| u_IL12p70_nom + | 114 | 137 | 40 | 50 | 0.203 | 0.800 | 0.832 | 0.847 |
| u_IL13_nom + | | | | | | | | |
| u_BTA | | | | | | | | |

TABLE 3
Biomarker combinations
Biomarker Combinations

BTA + Cystatin_B + EGF + IL12p70 + IL13 + Midkine + PAI_1tPA
BTA + Clusterin + Cystatin_B + EGF + IL12p70 + IL13 + Midkine + PAI_1tPA
BTA + Cystatin_B + EGF + IL12p70 + IL13 + Midkine + PAI_1tPA + Triglycerides
BTA + Cystatin_B + EGF + IL8 + IL12p70 + IL13 + Midkine + PAI_1tPA
BTA + Clusterin + Cystatin_B + EGF + IL12p70 + IL13 + Midkine + PAI_1tPA
BTA + Clusterin + Cystatin_B + EGF + IL12p70 + IL13 + Midkine + PAI_1tPA +
Triglycerides
BTA + Clusterin + Cystatin_B + EGF + IL12p70 + IL13 + Midkine + PAI_1tPA +
Triglycerides
BTA + Cystatin_B + EGF + IL12p70 + IL13 + Midkine + PAI_1tPA
BTA + Clusterin + Cystatin_B + EGF + IL12p70 + IL13 + Midkine + PAI_1tPA
BTA + Clusterin + Cystatin_B + EGF + IL8 + IL12p70 + IL13 + Midkine + PAI_1tPA
BTA + Cystatin_B + EGF + EGF + IL8 + IL12p70 + IL13 + Midkine + PAI_1tPA +
Triglycerides
BTA + Clusterin + EGF + IL12p70 + IL13 + Midkine + PAI_1tPA + Triglycerides
BTA + IL12p70 + IL13 + Midkine + PAI_1tPA
BTA + IL12p70 + IL13 + Midkine
BTA + IL12p70 + IL13 + Midkine + PAI_1tPA + Clusterin
BTA + IL12p70 + IL13 + Midkine + PAI_1tPA + Cystatin_B
BTA + IL12p70 + IL13 + Midkine + PAI_1tPA + EGF
BTA + IL12p70 + IL13 + Midkine + PAI_1tPA + EGF
BTA + IL12p70 + IL13 + Midkine + PAI_1tPA + Triglycerides
BTA + IL12p70 + IL13 + Midkine + Clusterin
BTA + IL12p70 + IL13 + Midkine + Cystatin_B
BTA + IL12p70 + IL13 + Midkine + EGF
BTA + IL12p70 + IL13 + Midkine + EGF
BTA + IL12p70 + IL13 + Midkine + Triglycerides
BTA + IL12p70 + IL13 + IL7 + PAI_1tPA
BTA + IL12p70 + IL13 + sTNFRI + PAI_1tPA
BTA + IL12p70 + IL13 + Cystatin_C + PAI_1tPA
CK18 + IL12p70 + IL13 + Midkine + PAI_1tPA
CK18 + IL12p70 + IL13 + IL7 + PAI_1tPA
CK18 + IL12p70 + IL13 + IL7
BTA + IL12p70 + IL13 + Midkine + PAI_1tPA + HdG80
BTA + IL12p70 + IL13 + Midkine + HdG80
BTA + IL12p70 + IL13 + Midkine + PAI_1tPA + CEA
BTA + IL12p70 + IL13 + Midkine + CEA
BTA + IL12p70 + IL13 + Midkine + PAI_1tPA + CK18
BTA + IL12p70 + IL13 + Midkine + CK18
BTA + IL12p70 + IL13 + Midkine + PAI_1tPA + Creatinine
BTA + IL12p70 + IL13 + Midkine + Creatinine
BTA + IL12p70 + IL13 + Midkine + PAI_1tPA + CXCL16
BTA + IL12p70 + IL13 + Midkine + CXCL16
BTA + IL12p70 + IL13 + Midkine + PAI_1tPA + Cystatin_C
BTA + IL12p70 + IL13 + Midkine + Cystatin_C
BTA + IL12p70 + IL13 + Midkine + PAI_1tPA + dDimer
BTA + IL12p70 + IL13 + Midkine + dDimer
BTA + IL12p70 + IL13 + Midkine + PAI_1tPA + FAS
BTA + IL12p70 + IL13 + Midkine + FAS
BTA + IL12p70 + IL13 + Midkine + PAI_1tPA + HAD
BTA + IL12p70 + IL13 + Midkine + HAD
BTA + IL12p70 + IL13 + Midkine + PAI_1tPA + IL1a
BTA + IL12p70 + IL13 + Midkine + IL1a
BTA + IL12p70 + IL13 + Midkine + PAI_1tPA + IL1a
BTA + IL12p70 + IL13 + Midkine + IL1a
BTA + IL12p70 + IL13 + Midkine + PAI_1tPA + IL1b
BTA + IL12p70 + IL13 + Midkine + IL1b
BTA + IL12p70 + IL13 + Midkine + PAI_1tPA + IL4
BTA + IL12p70 + IL13 + Midkine + IL4
BTA + IL12p70 + IL13 + Midkine + PAI_1tPA + IL6
BTA + IL12p70 + IL13 + Midkine + IL6
BTA + IL12p70 + IL13 + Midkine + PAI_1tPA + IL7
BTA + IL12p70 + IL13 + Midkine + IL7
BTA + IL12p70 + IL13 + Midkine + PAI_1tPA + IL8
BTA + IL12p70 + IL13 + Midkine + IL8
BTA + IL12p70 + IL13 + Midkine + PAI_1tPA + MCP_1
BTA + IL12p70 + IL13 + Midkine + MCP_1
BTA + IL12p70 + IL13 + Midkine + PAI_1tPA + Microalbumin
BTA + IL12p70 + IL13 + Midkine + Microalbumin
BTA + IL12p70 + IL13 + Midkine + PAI_1tPA + MMP9NGAL
BTA + IL12p70 + IL13 + Midkine + MMP9NGAL
BTA + IL12p70 + IL13 + Midkine + PAI_1tPA + MMP9TIMP1
BTA + IL12p70 + IL13 + Midkine + MMP9TIMP1
BTA + IL12p70 + IL13 + Midkine + PAI_1tPA + NGAL
BTA + IL12p70 + IL13 + Midkine + NGAL
BTA + IL12p70 + IL13 + Midkine + PAI_1tPA + NSE
BTA + IL12p70 + IL13 + Midkine + NSE
BTA + IL12p70 + IL13 + Midkine + PAI_1tPA + Progranulin
BTA + IL12p70 + IL13 + Midkine + Progranulin
BTA + IL12p70 + IL13 + Midkine + PAI_1tPA + TUP
BTA + IL12p70 + IL13 + Midkine + TUP
BTA + IL12p70 + IL13 + Midkine + PAI_1tPA + TGFb1
BTA + IL12p70 + IL13 + Midkine + TGFb1
BTA + IL12p70 + IL13 + Midkine + PAI_1tPA + Thrombomodulin
BTA + IL12p70 + IL13 + Midkine + Thrombomodulin
BTA + IL12p70 + IL13 + Midkine + PAI_1tPA + sTNFRI
BTA + IL12p70 + IL13 + Midkine + sTNFRI
BTA + IL12p70 + IL13 + Midkine + PAI_1tPA + TPA
BTA + IL12p70 + IL13 + Midkine + TPA
BTA + IL12p70 + IL13 + Midkine + PAI_1tPA + VEGF
BTA + IL12p70 + IL13 + Midkine + VEGF
IL12p70 + IL13 + Midkine
IL12p70 + IL13 + BTA

### REFERENCES

Fradet Y, et al., J Urol. 2007 July; 178(1):68-73, discussion 73.
Witjes J A, et al., Eur Urol. 2010 April; 57(4):607-14.
National Collaborating Centre for Cancer, Bladder Cancer: diagnosis and management; NICE Guidelines 2, February 2015, Page 78.
Van der Aa M N, et al., J Urol. 2010 January; 183(1):76-80.
Papa L, et al., Ann Emerg Med. 2012 June; 59(6):471-83.

1. A method for the detection of or the risk of bladder cancer in a female patient comprising the steps of
(i) detecting the presence of a panel of biomarkers in a sample isolated from a female patient, said panel of biomarkers comprising IL-13 and IL-12p70 and one or more biomarkers selected from BTA, Midkine, PAI-1/tPA, 8OHdG, CEA, CK18, Clusterin, Creatinine, CXCL16, Cystatin B, Cystatin C, d-Dimer, EGF, FAS, HAD, IL-1a, IL-1b, IL-4, IL-6, IL-7, IL-8, MCP-1, Microalbumin, MMP9NGAL, MMP9TIMP1, NGAL, NSE, Progranulin, TUP, TGFB1, Thrombomodulin, sTNFR1, TPA, VEGF and Triglycerides and/or the concentration of albumin/microalbumin/protein and creatinine expressed as an albumin:creatinine ratio;
(ii) assessing the presence or risk of bladder cancer in the female patient wherein detection of an elevated presence of the biomarkers compared to a normal control indicates the presence or the risk of cancer in the female patient from whom the sample is isolated.

2. The method of claim 1, wherein the one or more biomarkers are selected from BTA, Midkine, PAI-1/tPA, Clusterin, IL-8, Microalbumin, MMP9NGAL, NSE, Cystatin C, d-Dimer, IL-7.

3. The method of claim 1, wherein the panel of biomarkers comprises BTA.

4. The method of claim 1, wherein the panel of biomarkers comprises Midkine.

5. The method of claim 3, wherein the panel of biomarkers comprises PAI-1/tPA.

6. The method of claim 1, wherein the panel of biomarkers is selected from the combination of biomarkers in Table 2 or Table 3.

7. The method of claim 6, wherein the panel of biomarkers is selected from:
(i) BTA, IL-13 and IL12p70;
(ii) Midkine, IL-13 and IL12p70;
(iii) BTA, IL-13, IL12p70 and Midkine; and
(iv) BTA, IL-13, IL12p70, Midkine and PAI-1/tPA.

8. The method of claim 1, wherein one or more of the biomarkers are the urinary form.

9. The method of claim 8, wherein each biomarker is the urinary form.

10. The method of claim 1, wherein the method further comprises a step of characterizing the patient's infection status.

11. The method of claim 1, wherein said sample is a urine sample.

12. The method of claim 1, wherein step (ii) comprises inputting the measured concentrations of the biomarkers from step (i) into an algorithm such that the output of the algorithm indicates whether the individual has or is at risk of developing bladder cancer.

13. The method of claim 12, wherein the output of the algorithm has a sensitivity of at least 0.70.

14. The method of claim 12, wherein the output of the algorithm has a specificity of at least 0.70.

15. The method of claim 1, wherein the patient has exhibited haematuria.

16. A solid support material comprising binding molecules attached thereto, said binding molecules having affinity specific for IL-13 and, separately, IL-12p70, with the binding molecules for each being in discrete locations on the support material.

17. The solid support material according to claim 16, further comprising, each in discrete locations, binding molecules for one or more of the additional biomarkers.

18. The solid support material according to claim claim 17, wherein the binding molecules, separately, have affinity for the biomarkers defined in Table 2 or Table 3.

19. The solid support material according to claim 18, wherein the binding molecules, separately, have affinity for the biomarkers:
(i) BTA, IL-13 and IL12p70;
(ii) Midkine, IL-13 and IL12p70;
(iii) BTA, IL-13, IL12p70 and Midkine; and
(iv) BTA, IL-13, IL12p70, Midkine and PAI-1/tPA.

20. The solid support material according to claim 16, wherein the binding molecules are antibodies.

21. The solid support material according to claim 16, wherein the support is a biochip.

22. A method for the detection of or the risk of bladder cancer in a female patient comprising the steps of
(i) determining that a female patient does not have an infection;
(ii) detecting the presence of one or more biomarkers in a sample isolated from the female patient, wherein said one or more biomarkers are selected from IL-13, IL12p70, BTA and Midkine;
(iii) assessing the presence or risk of bladder cancer in the female patient wherein detection of an elevated presence of the biomarkers compared to a normal control indicates the presence or the risk of cancer in the female patient from whom the sample is isolated.

23. The method according to claim 22 wherein said one or more biomarkers are selected from the following:
(i) IL-13+IL12p70
(ii) IL-13+BTA
(iii) IL-13+Midkine
(iv) IL12p70+BTA
(v) IL12p70+Midkine
(vi) BTA+Midkine.
